(19) United States
(12) Patent Application Publication
(10) Pub. No.: US 2008/0236600 A1
(43) Pub. Date: Oct. 2, 2008

(54) Nail biting deterrent device and method

(76) Inventor: Donald L. Davinroy, Collinsville, IL (US)

Correspondence Address: DON W. WEBER, 235 BRISTOL, TROY, IL, 62294, US

(21) Appl. No.: 11/728,300

(22) Filed: Mar. 26, 2007

(51) Int. Cl. A61F 5/37

(52) U.S. Cl. 128/880

(57) ABSTRACT

A partial mouthpiece is made of soft, flexible dental material and is manufactured to conform to the shape and size of a user's lower front biting teeth. The preferred mouthpiece would cover the four lower incisors and two canine teeth, but an acceptable alternative would also cover the lower left and right first molars as well. The mouthpiece is thin and made to conform to the arcuate shape and size of a user's own teeth. The mouthpiece has a thickness of approximately 0.037 inches. The thin structure of the mouthpiece prevents the user from biting his nails, but does not inhibit speech, is hardly noticeable when in use and is aesthetically pleasing. To use the device, the nail biter creates a list of events that have a tendency to trigger nail biting. The mouthpiece is inserted onto the subject's lower front biting teeth whenever the urge to bite fingernails arises. The partial mouthpiece prohibits the subject from biting his nails and the habit is eventually broken.

### BACKGROUND OF THE INVENTION

This invention relates to the field of personal hygiene and habit breaking. More particularly, a dental mouthpiece is provided which helps enable the user to stop biting his fingernails.

Fingernail biting is a chronic problem throughout the United States and the world. While the problem is most prevalent in adolescent children, it is common in adults as well. It is estimated that 30% of children ages 7 to 10, 44% of adolescent children, 25% of young adults and 5% of adults are nail biters. Problem nail biting is widespread and common. Approximately 20% of the adult population is at any one time a nail biter, with rates among college students being much higher at 30%. One of the more noteworthy and generally surprising aspects of nail biting is that is crosses every social and economic barrier.

Nail biting is a serious social and health issue. The results of nail biting can result in short, ragged nails. It may also lead to damaged cuticles as well as bleeding around the edges of nails. Infections can also develop if damaged nails are not properly treated. Psychological effects include a lack of self-esteem, embarrassment and a feeling of inadequacy. Nail biting is a habit, not a disease. Its medical term is onychophagia. Experts in the field have stated bluntly that there is clearly no effective treatment for onychophagia.

Due to the prevalence of this habit, many different types of cures and treatments have been tried and used, mostly to little or no effect. For example, nasty tasting creams and polishes have been suggested to deter nail biting. This method has been known to deter nail biting for a few weeks, but a person who is truly habituated to nail biting will learn how to get around that method. Acupuncture, prayer groups, blogs, message boards, self help books, psychotherapy, counseling, omega-3 treatments, audiotapes, self-therapy and even hypnotism have been tried, often with mixed results.

Several United States patents have been filed directed to the problem of breaking the habit of nail biting. The 1990 U.S. Pat. No. 4,976,275 issued to Dixon is one such example. Dixon disclosed a device that is placed in one's mouth and that prevents the user's tongue from contacting the nail. It was thought that preventing this sensation will help alleviate the habit. However, the user was still able to bite the nails even if the Dixon device were in place. It is an object of the present invention to provide an anti-nail biting device that prohibits the user from biting his nails when the device is deployed.

Another method and device for preventing nail biting is found in US patent application published in October of 2006, US 2006/0219251. The device described in that publication included an artificial thumb used to decondition the user from the habit. Such a device is cumbersome and socially unattractive. It is a further object of this invention to provide an anti-nail biting device that is virtually undetectable when in use and which would not draw undue attention to its use when deployed.

The instant device places the focus of the cure to habitual nail biting on the inability of the user to complete the bite. The essence of the structure of the device is to provide a thin, clear tooth-covering device that prevents the user from biting his fingernails when the device is in use. This device differs from the well-known athletic or dental tooth covering or protecting device currently in use. Standard athletic mouthpieces are used for a different purpose and hence are structurally different from the instant device.

Most athletic mouth guards have a cross-section (thickness) of approximately 0.130 inches (3.3 mm) more or less. For example, U.S. Pat. No. 6,986,354, issued to Burns, discloses a mouth guard that provides a shield from blows received in athletic competition. However, the Burns mouth guard is thick at the front to provide a labial shield and has a flat wide upper surface. A thick mouth guard designed for use to prevent nail biting would inhibit correct pronunciation and would be cosmetically obvious. It is still further object of this invention to provide a cosmetically non-obvious mouth guard for the prevention of nail biting that does not inhibit pronunciation when deployed.

One final aspect of this invention is its attention to the aesthetic aspects of a mouthpiece. Typically, mouth guards are not aesthetically designed and hence are obvious social deterrents. U.S. Pat. No. 6,830,051 issued to Lesniak is a good example of a functional mouthpiece that is not aesthetically pleasing. The typical Lesniak interocclusal appliance has thick walls and flat outer surfaces. The Lesniak device has walls that are at least 4 mm (0.160 inches) thick to insure proper protection from blows to the teeth or falls. The top of the lower and the bottom of the upper pieces are flat, with widths of approximately 0.080 inches or greater. Both of these features (the thickness of the walls and the flat wide upper and lower surfaces) provide a mouthpiece that is functional as a teeth protector but is not aesthetically pleasing or socially acceptable except when playing the sport. It is a still and further object of this invention to provide an aesthetically pleasing and socially acceptable mouthpiece for breaking the nail biting habit that may be worn or deployed at all times and still perform its habit-breaking function. Further, a method of use of the device is also disclosed which provides maximum habit-breaking potential at a minimum social cost.

Other and further and other objects of this invention will become obvious upon reading the below described specification.

### BRIEF DESCRIPTION OF THE DEVICE

A v-shaped dental insert for the bottom 6 to 10 teeth is fitted by various methods to cover a user's lower incisors, canine and first molars. The insert is clear and thin. The insert conforms to the shape of the user's lower teeth and is virtually unnoticeable when deployed. The habitual nail biter is instructed to make a list of the times when he is most likely to bite his nails, such as while driving, watching television or movies, reading, or taking tests. The nail biter places the device over the user's lower teeth whenever the user is in danger of biting his nails. Due to the thin, clear structure of the device, it does not inhibit speech and is not visually obvious.

### BRIEF DESCRIPTION OF THE DRAWING FIGURES

FIG. 1 is a perspective view of the device.

FIG. 2 is a perspective view of the standard athletic mouth guard.

FIG. 3 is a cross-sectional perspective view of the device taken along lines 3-3 of FIG. 1 showing the narrow width of the walls.

### DETAILED DESCRIPTION OF THE PREFERRED EMBODIMENT

Many methods have been attempted to help break the habit of nail biting. However, the methods of the prior art all fail to focus on the key element of the habit—the bite. The disclosure of this invention directs itself to the bite, rather than to the causes of the habit. If a nail biter has a willingness to try to break the habit, this aid will enable the person to break the habit. If the mouthpiece is in place, covering the bottom teeth as disclosed, it is virtually impossible to make the bite. This impossibility leads to the habit being broken in a matter of weeks or at the very most several months.

In order to bite one's fingernails, the front teeth of the biter are utilized. The six upper front “biting” teeth comprise the central incisors, the lateral incisors and the canine teeth. The lower front biting teeth comprise the four lower incisors, the flanking canine teeth, and, to a lesser extent, left and right first molars. In order to prevent the biting of nails, only the lower six to eight biting teeth need be covered with a partial mouthpiece. It has been found in practicing this invention that the lower front teeth are most suitable for receiving the mouthpiece. However, it is also within the spirit of this invention that a thin mouthpiece as described below be utilized on the upper 7 to 10 teeth.

The present invention involves two new and novel disclosures. The first concept is the thin, clear aesthetically pleasing partial mouthpiece covering. The second concept is the method of use of the mouthpiece.

The nail bite stopper partial mouthpiece consists of a thin covering for the lower front six or eight teeth. It is to be appreciated that at least six lower front teeth (lower incisors and canine teeth) must be covered to prevent nail biting. However, it is within the contemplation of this invention that the eight front, lower teeth be covered by the device (incisors, canine and first molars), or that the upper teeth be covered.

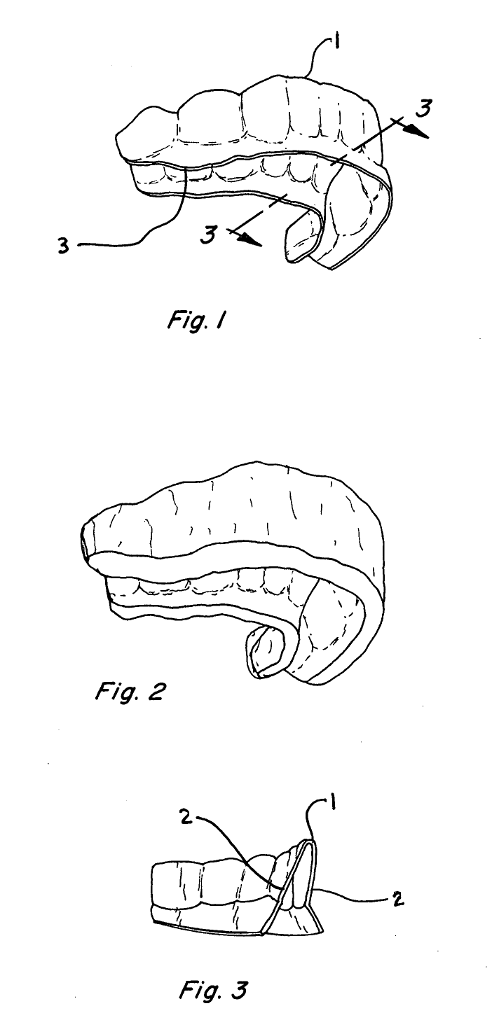

The partial mouthpiece has a generally inverted V-shaped cross-section as shown in FIG. 1. The mouthpiece is described as inverted “V” shaped rather than “U” shaped to emphasize the thin top upper surface of the device. The device is not flat on the upper surface (U-shaped) but rather is thin (V-shaped).

The inverted V-shape is arcuate and conforms to the general shape of a lower dental arch. The partial mouthpiece shown in FIG. 1 has an upper surface 1 that is in contact with the upper surface of the user's front lower teeth. The structure also has lower downwardly and outwardly sloping sides 2. The slight slope helps to keep the partial mouthpiece in place and in contact with the user's lower front teeth.

The upper surface of the inverted V-shaped mouthpiece is only slightly thicker than the tooth itself. In the preferred embodiment, the thickness 3 of the top surface 1 and sides 2 of the nail bite stopper mouthpiece is approximately 0.037 inches (approximately 1 mm).

The overall shape of the partial mouthpiece is generally trough-shaped as shown in FIGS. 1 and 3. The wall thickness 3 of the entire partial mouthpiece is critical due to the nature of the device. The thickness of the instant mouthpiece may not be more than 0.050 inches, with 0.037 inches being preferred. The upper surface 1 of the mouthpiece should conform to the shape of the upper surface of the lower front teeth and should be V-shaped, not flat or otherwise distinctive.

While it is anticipated that the covering be clear, it also within the spirit and disclosure of this invention to have colored nail bite stopper mouthpieces. In the event the nail bite stopper becomes more acceptable in society, or in the event that the end user is particularly confident or gregarious, colored mouthpieces would be an option.

In the instant invention, a thin-walled, clear, aesthetically pleasing and inconspicuous mouthpiece is desired. The thickness of the walls of the instant mouthpiece should be approximately 0.037 inches or less and not greater than 0.050 inches. The preferred embodiment of the device has a top surface and wall thickness that is at least 0.025 inches but not greater than 0.050 inches.

The very narrow thickness of the instant device is distinguished from the labial walls of the Lesniak mouthpiece that is approximately 3 to 4 mm (0.120-0.160 inches). Further, the instant mouthpiece has no reinforcing or strengthening material as distinguished from the prior art, such as the thickened forward labial shield disclosed in Burns (U.S. Pat. No. 6,986,354).

Finally, the instant mouthpiece is shaped to conform to the general arcuate shape of a user's lower front teeth (generic) or to the exact shape of the customized user's lower teeth. The nail bite stopper mouthpiece is not flat on top as shown in the prior art and covers only the lower six to eight teeth of the user.

It is to be appreciated that only the lower front six or eight teeth need be covered to practice this invention. While it is preferred to cover only the lower four incisors and two canine teeth, a minor variation would be to cover the left and right first molars as well.

The manufacture of the actual mouthpiece can be done by any conventional method. However, the essence of this invention is the recognition that only the lower six to eight teeth need be covered to practice this invention. Many different methods of manufacture of conventional mouthpieces are well known in the art and have been fully described in the literature.

The best-known and preferred method of manufacture utilizes injection molding. For example, the Lesniak '051 patent specification describes examples of methods of manufacture and materials used to make flat, thick mouthpieces designed for use in preventing bruxism. Cook, in U.S. Pat. No. 6,820,623 also describes the manufacture of a customizable dental appliance for use in athletics, to prevent bruxism, for bleaching of teeth and to help control breathing and snoring. The general methods of manufacture disclosed in Lesniak, Cook and other patents and publications could all be utilized in practicing this invention. However, distinct differences in the final product distinguish this invention over the prior art mouth guards due to the necessary thin width of the walls as described above.

It has been found in clinical trials that the covering of the lower front teeth with a thin, clear covering may be accomplished utilizing different materials. Unlike other mouthpieces, the materials should yield a soft yet slightly flexible and should be molded to conform to a user's lower front biting teeth. Some of the more favored dental materials include EVA (ethylene vinyl acetate), flexible acrylic 40D, EVA polymer plastic, single shot thermoplastic, inert dental grade plastic, thermo plastic, pliable thermo rubber, medical grade silicone, moldable plastic, dental vinyl PVC, synthetic rubber approved for use by the FDA, low density polyethylene with a tactifier resin, PTFE with electrometric coating or moldable thermoplastic.

It is contemplated that mouthpieces may be made in one generic size, in three different general sizes (Small, Medium, Large) or customized to fit the individual mouth. In making a general sized mouthpiece (S, M, L) approximately three standardized mouthpieces can be manufactured in bulk. The end user of the product would simply measure the distances between the left and right canine teeth. A general, standardized, sized (S, M, L) mouthpiece could then be ordered. These standardized sizes can be sized according to dental tables listing standard sizes of lower front dental arches.

It is also within the contemplation of this disclosure that a boil-and bite kit could be sent to the user. Boil-and-bite kits are well known in the mouthpiece art. Utilizing this process, a thin layer of moldable material would be sent to the end user. The user would heat the material by boiling it in hot water until it softens. Then the user would simple place the heated and softened material on his lower front teeth and bite down. The mouthpiece is then removed and allowed to harden to some degree. The mouthpiece is then permanently shaped to correspond to the user's specific lower front teeth.

Alternatively, a customized mouthpiece could be prepared. To prepare a customized mouthpiece, the user would follow the usual steps in preparing dental impressions. However, only the dental impression of the lower six or eight teeth would be necessary. A dental impression kit would be sent to the individual. Putty would be mixed and placed into the dental tray. The putty in the tray would then be placed over the lower front dental arch, covering the lower incisors, canine and first molars. After a few minutes the tray is removed and the dental impression of the required teeth will be sent back to the lab where the nail bite-stopping mouthpiece will be manufactured.

The instant mouthpiece has two important aspects. The first is to prevent one from biting one's nails. The second, and as important, is to provide an unobtrusive, aesthetically pleasing mouth guard that would be utilized in social as well as private situations. The structural differences between the instant device (wall thickness, shaped to conform to teeth, narrow top surface, only lower front teeth) are required to practice this invention because of the unique need to provide a thin, aesthetically pleasing appearance to satisfy this two-fold function.

Once the nail bite stopper mouthpiece is available, a proper method of use is also required to break the nail biting habit. The end user would first make a list of events when fingernail biting is likely to occur. These events vary from individual to individual but cold include watching television, movies or sporting events, using the computer, taking tests, driving, or working at a desk. The user should keep the bite stopper with him or her at all times until the habit is broken. Whenever the user feels the urge to bite the fingernail, the nail bite stopper is inserted into the mouth and onto the lower teeth. The user is then absolutely prohibited from biting the fingernail and is constantly reminded of his inability to bite. The mouthpiece should be kept deployed until the urge to bite the nails has passed. After several weeks or a few months of usage, the nail biting habit is broken.

It is to be emphasized that the nail bite stopper and method disclosed herein is only an aid to break the habit of nail biting. The user must be willing to use the mouthpiece whenever the urge to bite is present and the user must persevere. However, it has been shown in clinical tests that the use of the nail bite stopper, in conjunction with the list of events and deployment of the device, is an effective means to break the habit of fingernail biting.

Having fully described my device, I claim:

1. A partial mouthpiece for covering the front teeth of a user, comprising a thin, arcuate partial mouthpiece with a V-shaped cross-section having an upper surface and lower downwardly and outwardly sloping sides adapted to receive the front biting teeth of a user.

2. A partial mouthpiece as in claim 1, wherein the V-shaped partial mouthpiece is manufactured in standardized dental small, medium and large sizes.

3. A partial mouthpiece as in claim 1, wherein the cross-section of the device is between 0.025 and 0.050 inches.

4. A partial mouthpiece as in claim 1, wherein the cross-section of the device is 0.037 inches.

5. A partial mouthpiece as in claim 4, wherein the upper surface of said partial mouthpiece conforms to the shape of the upper surface of the user's lower front teeth.

6. A partial mouthpiece as in claim 5, wherein said partial mouthpiece is made of clear, aesthetically pleasing soft dental material.

7. A method utilized to break a user of the nail biting habit, comprising:
(1) Making a list of events where the user is likely to have the urge to bite one's nails;
(2) Placing the device described in claim 1 onto the user's lower, front biting teeth when the urge to bite fingernails is acquired;
(3) Leaving the partial mouthpiece deployed until the urge to bite one's fingernails subsides.
